(19) United States
(12) Patent Application Publication
(10) Pub. No.: US 2025/0025214 A1
(43) Pub. Date: Jan. 23, 2025

(54) SPINE STABILIZATION

(71) Applicant: MOVING SPINE AG, ZÜRICH (CH)

(72) Inventors: Jonas WIDMER, Fällanden (CH); Mazda FARSHAD, Zumikon (CH); Robert FRIGG, Bettlach (CH)

(73) Assignee: MOVING SPINE AG, ZÜRICH (CH)

(21) Appl. No.: 18/224,679

(22) Filed: Jul. 21, 2023

(51) Int. Cl. A61B 17/70; A61B 17/84

(52) U.S. Cl. CPC A61B 17/7053; A61B 17/842

(57) ABSTRACT

Vertebral stabilization techniques labelled “vertebropexy” can be used after microsurgical decompression (intact posterior structures) and midline decompression (removed posterior structures). Vertebropexy involves semi-rigid spinal stabilization based on reinforcement of the spinal segment and is able to reduce motion, especially in flexion-extension.

### BACKGROUND OF THE INVENTION

### Field of the Invention

The present invention relates to methods of stabilizing the spine.

### Discussion of Related Art

The biomechanical understanding of the human body is of utmost importance in orthopedic surgery and especially in spine surgery. When degeneration occurs, the stability of the disc and ligaments decreases, which can lead to instability of the segment and thus pain.

Spinal fusion has become a very common surgical procedure, among others in the treatment of degenerative disorders of the spine. The indications for this surgical procedure are diverse and include low-back pain due to facet joint osteoarthritis, degenerative spondylolistheses, degenerative scoliosis and segmental instability. The latter can also be a result of iatrogenic destabilization following surgical resection of ligamentous structures as well as the facet joint. However, spinal fusion is associated with serious long-term complications such as adjacent segment degeneration (ASD), screw loosening, pseudarthrosis, implant failure, and, in rare cases, neurovascular injury during implant insertion [1-5]. The redistribution of loads with subsequently increased biomechanical stress are believed to act as accelerators of ASD [1, 2, 6] and proximal junctional kyphosis [7]. Further, long fusions can lead to a relevant, irreversible loss of motion, which can cause postural changes [8].

Despite these problems, posterior spinal fusion (PSF) currently represents the gold standard. Alternative techniques of spinal stabilizations have not yet yielded satisfactory results with broad clinical impact. Semi-rigid fixation techniques have been proposed to overcome the above-mentioned challenges, but resulted in new complications at the implant-bone interface such as device breakage, dislocation or screw loosening [9-11]. The previous attempts at spinal stabilization also include spinous process implants. However, studies have shown higher rates of reoperation with low cost-effectiveness, which may explain why they are hardly in use anymore [9, 11]. The same applies to cervical wiring techniques, such as sublaminar wires for atlantoaxial fusion first described by Galli in 1939, which are unable to achieve sufficient stabilization.

### SUMMARY OF THE INVENTION

In conclusion, despite continued efforts to achieve appropriate spine stabilization, no technique has gained clinical acceptance. A major shortcoming of the known techniques is insufficient stabilization of the spine, in particular in the long term. A further shortcoming is that the known techniques frequently lead to significant, sometimes complete, immobilization of the segment, in particular in clinically important directions such as flexion-extension and shear movement. Therefore, there is a need to advance the state of the art with respect to stabilization of the spine.

The present disclosure is aimed at providing a method to stabilize the spine of a patient in need thereof, while at least partially avoiding the complications associated with the known techniques, particularly the implant-and fusion-related complications. In particular, it is an object of the present disclosure to achieve targeted stabilization of the spine without immobilizing the segment. With respect to the directions of motion, the present disclosure aims to achieve passive stability in clinically important directions of motion such as flexion-extension and shear movements, but without stiffening other directions of motion. It is a further object to provide a reversible method of spine stabilization. It is a further object for at least some embodiments to improve the biological compatibility of the implanted material used in the method, e.g., by enhancing the biological integration of the implanted material into the surrounding tissue.

According to the present disclosure, these objects are addressed by the features of the independent claims. In addition, further advantageous embodiments follow from the dependent claims and the description.

The present disclosure relates to a method of stabilizing the spine of a patient in need thereof.

a) The method comprises the step of providing a first vertebra having a first fastening edge and a second vertebra having a second fastening edge, wherein the first fastening edge and the second fastening edge are arranged opposite each other and facing away from each other.

b) The method further comprises the step of providing a strap extending in a longitudinal direction from a front section to a rear section.

c) The method further comprises the step of passing the front section of the strap around the first fastening edge, then from the first fastening edge to the second fastening edge and then around the second fastening edge.

d) The method may further comprise the optional step of optionally passing the front section of the strap from the second fastening edge to the first fastening edge and repeating step c).

e) The method further comprises the step of tensioning the strap such that the first vertebra and the second vertebra are fixated relative to each other.

f) The method further comprises the step of fastening the front section to the rear section while the strap is being tensioned.

The method of the present disclosure may be labelled as “vertebropexy” and it constitutes a new concept of semi-rigid spinal stabilization based on reinforcement of the spinal segment using a strap, such as a ligament or a synthetic material. Vertebropexy not only restores the native segmental stability after decompression, but also transfers the segment to a semi-rigid state.

The method of the present disclosure and the variants disclosed herein have a range of advantages. They allow a targeted stabilization of the spine to counteract degeneration-related or iatrogenic (e.g., decompression) instability, but without immobilizing the segment. Depending on the application, native stability of the segment may be restored fully or partially after surgical decompression and the segment may be placed in a more stable state, without complete immobilization. Depending on the application, lumbar segmental motion may be significantly reduced, especially in flexion-extension and, though typically to a lesser extent, in shear motion. Depending on the variant of the method being used, all other directions of motion may remain flexible and correspond to the preoperative range of motion. The method of the present disclosure may preferably be reversible, thereby still allowing traditional techniques such as spinal fusion to be performed subsequently. The method may also be carried out using no foreign material, e.g., using ligaments or allografts, which enhances the biocompatibility and may allow the implanted material to grow together with adjacent tissue. Furthermore, the method of the present disclosure may be used for different indications: for example, an interspinous variant can be used after microsurgical decompression if the surgeon wishes to achieve more stability, such as in existing low-grade spondylolisthesis. When the posterior structures are omitted, such as after midline decompression, a spino-laminar variant can be used. Finally, the technique disclosed herein allow for an easy handling by the surgeon.

The strap may, for example, be able to undergo deformations elastically, without permanent damage. Depending on the application, the strap may be hyperelastic. Additionally, or alternatively, depending on the application, the strap may be viscoelastic. As an example, the strap may have an ultimate load of at least 1700 N, preferably from 2000 N to 5000 N. The ultimate load is typically defined as the maximum load that the strap can endure without tearing. In some variants, the strap may have a linear stiffness from 320 N/mm to 520 N/mm, preferably from 370 N/mm to 470 N/mm. Additionally, or alternatively, the strap may have an ultimate displacement of at least 6 mm, preferably from 7 mm to 14 mm. The parameters described in this paragraph refer to a strap length of 95 mm. The parameters described in this paragraph may, for example, be measured as described in the third example below.

The method of the present disclosure includes the step of e) tensioning the strap. The tensioning may, for example, lead to the first vertebra and the second vertebra being positioned relative to each other. In some variants, the first vertebra and the second vertebra may be at least partially fixated relative to each other. Depending on the application, the strap may be tensioned in different ways and with various effects. For example, the strap may be tensioned such that the first vertebra and the second vertebra are fixated relative to each other with respect to at least one direction of motion. In other words, motion of the first vertebra relative to the second vertebra may at least partially be restricted in at least one direction of motion. Depending on the field of application, one or more directions of motion may be affected. In some variants, relative movement between the first vertebra and the second vertebra may be at least partially restricted in one or more of the following directions of motion: flexion, extension, lateral bending, axial rotation, anteroposterior shear and lateral shear. The relative movement in the respective directions of motion may be restricted to different degrees. As an example, in one variant, the range of motion in flexion and/or extension is decreased by at least 50%, preferably by at least 60%, compared to the native state. The native state describes the state of the first vertebra and the second vertebra before performing the method disclosed herein.

Depending on the application, one of the first vertebra and the second vertebra may be labelled as a cranial vertebra and the other of the first vertebra and the second vertebra may be labelled as a caudal vertebra. The first vertebra and the second vertebra are typically adjacent to each other, specifically directly adjacent to each other.

Depending on the application, the step of providing the first vertebra and the second vertebra may include one or more preparatory surgical steps. The preparatory surgical steps may include a laminotomy, optionally followed by flavectocomy, optionally followed by recessotomy. Additionally, or alternatively, the preparatory surgical steps may include removal of supraspinous and interspinous ligaments. Additionally, or alternatively, the preparatory surgical steps may include at least partial removal of the spinous processes. Depending on the application, the first vertebra may already include the first fastening edge in its native state, i.e., prior to performing the method. Additionally, or alternatively, the second vertebra may already include the second fastening edge in its native state. In further variants, it may be necessary to provide the first fastening edge and/or the second fastening edge, e.g., by means of a surgical step such as boring, drilling or cutting. As an example, step a) may include drilling a hole in the spinous process of the first vertebra and/or the second vertebra. The hole in the spinous process may e.g., have a diameter from 2 mm to 10 mm, preferably from 4 mm to 6 mm. Drilling the hole may include pre-drilling a pre-hole and then overdrilling the pre-hole to provide the hole. The pre-hole may for example have a diameter from 1 mm to 5 mm, preferably from 3 mm to 3.5 mm.

When carrying out the method of the present disclosure, typically, access is provided to a proximal side of the first vertebra and the second vertebra. Depending on the application, the proximal side may for example be a dorsal side of the vertebrae and the distal side may be oriented towards the vertebral foramen. Step c) may include passing the front section of the strap from a proximal side around the first fastening edge to a distal side, then on the distal side from the first fastening edge to the second fastening edge and then from the distal side around the second fastening edge to the proximal side. Optionally, step d) may then include passing the front section of the strap on the proximal side from the second fastening edge to the first fastening edge and repeating step c).

Depending on the application, the front section may be fastened to the rear section in different ways. Typically, the front section is connected to the rear section such that it directly contacts the second section. In some variants, the fastening includes at least one of knotting, suturing, sewing, stitching, knitting or felting the front section to the rear section. Preferably, the fastening includes suturing or knotting the front section to the rear section. In some variants, the front section is fastened to the rear section such that the front section and the rear section are approximated towards each other, which may involve application of a tensile force to the front section in a direction towards the rear section and application of a tensile force to the rear section in a direction towards the front section. When the fastening involves knotting, typically, two or more knots are knotted, preferably from four to seven knots.

The strap may be made of different materials. For example, the strap may comprise a synthetic material, a ligament or a tendon, preferably a ligament. The ligament may, for example, be an autograft, an allograft or a xenograft. Preferably, the ligament is an allograft. The synthetic material may, for example, be a non-metallic cerclage. When using a non-metallic cerclage, for example, an Arthrex FiberTape Cerclage may be used. Depending on the application, the non-metallic cerclage may be braided from a polyblend of ultra-high molecular weight polyethylene and polyester materials. Additionally, or alternatively, the non-metallic cerclage may be a flat braided suture.

One advantage of using a ligament or a tendon is that it ensures biological compatibility and allows the strap to grow together with surrounding tissue. One advantage of including a synthetic component is that synthetic components are typically widely available and relatively cheap compared to biological material.

Depending on the application, the strap may or may not comprise additional components in addition to a main body. For example, the strap may comprise reinforcement elements connected to the main body. Additionally, or alternatively, the strap may comprise a front fastening fiber connected to a main body of the strap in or near the front section and a rear fastening fiber connected to the main body of the strap in or near the rear section, wherein the rear fastening fiber forms a loop configured for receiving the front fastening fiber. The front fastening fiber and/or the rear fastening fiber may, for example, each be connected to the main body of the strap by a stitch, preferably a Krackow or Baseball stitch. Typically, the front fastening fiber and/or the rear fastening fiber are each connected to the main body of the strap in a tensile-resistant manner.

The front fastening fiber and the rear fastening fiber may for example serve to facilitate the step of tensioning the strap. In an embodiment, the step of tensioning the strap comprises creating a tension knot between the front fastening fiber and the rear fastening fiber, tensioning at least the front fastening fiber and tightening the tension knot. Optionally, the step of tensioning the strap may include applying opposite tensile forces to the front fastening fiber and the rear fastening fiber.

The method disclosed herein may be performed in different vertebrae and different sections, regions or areas of the vertebrae.

In a first variant, which may be labelled as “interlaminar vertebropexy”, one of the two vertebrae is a cranial vertebra and the other of the two vertebrae is a caudal vertebra, wherein the fastening edge of the cranial vertebra is a cranial edge of the lamina and the fastening edge of the caudal vertebra is a caudal edge of the lamina. In it, the distance in the longitudinal direction between a front end of the front section and a front end of the rear section of the strap in a relaxed state may, for example, range from 120% to 180%, preferably from 140% to 160%, more preferably 150%, of the distance between the first fastening edge and the second fastening edge.

In a second variant, which may be labelled as “interspinous vertebropexy”, one of the two vertebra is a cranial vertebra and the other of the two vertebra is a caudal vertebra, wherein the fastening edge of the cranial vertebra is a hole drilled in the spinous process of the cranial vertebra and the fastening edge of the caudal vertebra is a hole drilled in the spinous process of the caudal vertebra. In it, the distance in the longitudinal direction between a front end of the front section and a front end of the rear section of the strap in a relaxed state may, for example, range from 300% to 500%, preferably from 350% to 450%, more preferably from 380% to 420%, of the distance between the first fastening edge and the second fastening edge.

In a third variant, which may be labelled as “spinolaminar vertebropexy”, one of the two vertebra is a cranial vertebra and the other of the two vertebra is a caudal vertebra, wherein the fastening edge of the cranial vertebra is a cranial edge of the lamina and the fastening edge of the caudal vertebra is a hole drilled in the spinous process of the caudal vertebra.

Depending on the application, the strap may be applied such that tensile forces are applied to the vertebrae in different directions. For example, after fastening the front section to the rear section, the strap may exert a tensile force on the first vertebra and on the second vertebra in a tensile force direction that is essentially in parallel to the direction of extension of the spine between the first vertebra and the second vertebra. This direction may, for example, be used for interspinous vertebropexy or intervertebral vertebropexy. In a further variant, after fastening the front section to the rear section, the strap may also exert a tensile force on the first vertebra and on the second vertebra in a tensile force direction that includes a vector component in the dorsal direction. This direction may, for example, be used for spinolaminor vertebropexy. These variants may be used for different applications, depending on the needs of the patient. For example, providing a tensile force that is essentially in parallel to the direction of extension of the spine may lead to a particularly high stabilization of flexion-extension while still allowing mobility in other directions of movement, in particular shear movements. On the other hand, providing a tensile force that includes a vector component in the dorsal direction typically has a greater effect on shear motion.

Depending on the integrity of the dorsal structures after decompression, different variants may be used. As an example, the variant labelled “interspinous vertebropexy” may be used following posterior microsurgical decompression with preservation of midline structures. Additionally, or alternatively, the variant labelled “interlaminar vertebropexy” may be used following posterior decompression without preservation of midline structures. These structures mostly provide passive stability in flexion.

The variant labelled as “spinolaminar vertebropexy” allows for a range of advantages. Among them, this variant affects shear forces to a particularly great extent. Without wishing to be bound to a theory, this could possibly be explained by the fact that the fixation of the segment with the spinolaminar technique happens not purely in the cranio-caudal direction, but also in the antero-posterior direction and can thus absorb forces in this direction. Further advantages include an increase in a-p stability and easy tensioning with designated tensioning systems.

The tensioning and fastening may be performed in different ways and using different forces. In some variants, during the step of tensioning the strap, a tension force from 40 N to 90 N, preferably from 50 N to 70 N, is applied. The fastening may include knotting a knot. In further variants, the step of fastening the front section to the rear section may include overlapping the front section with the rear section and connecting the front section with the overlapped rear section. Typically, the front section is overlapped with the rear section such that the front section and the overlapped rear section extend in opposite directions. Depending on the application, the front section may for example be connected with the overlapped rear section by suturing, knitting or felting, preferably suturing. The region of overlap may, for example, have a length from 5 mm to 35 mm.

One advantage of the method of the present disclosure is that it allows for durable mechanical stabilization of the spine. For example, when the front section and the rear section are overlapped in opposing directions, the front section and the rear section may continue to extend along their previous directions, and there is no need to form a folded edge to join the two ends together, which would be necessary if the front section and the rear section were inserted into a clamping element serving to connect the two sections. Since no folded edge is formed, the risk of generating a crack or tear in the strap is minimized.

A further advantage is that in its implanted stage, the strap may only occupy a small volume and therefore constitute a minimal intrusion to the body. This is possible because the strap is first tensioned and then the front section is fastened to the rear section. As an example, it is not necessary to use an additional clamping element that clamps the front section and the rear section in order to fasten them. Rather, it is possible, for example, to knot, suture, sew, stitch, knot or felt the front section to the rear section. Thereby, no additional body volume is occupied by an additional clamping element. Depending on the application, the strap may have different geometries. In some variants, at a position of the strap in which the front section has been fastened to the rear section, the thread has a maximum width of up to 10 mm, preferably up to 8 mm, more preferably up to 6 mm, in at least one of the following directions: dorsal direction or lateral direction. For example, the dorsal extension may be less than 6 mm. Additionally, or alternatively, the area occupied by the strap in a cross sectional area orthogonal to the direction of extension of the spine between the first vertebra and the second vertebra may, for example, not exceed 100 mm2, preferably may not exceed 60 mm2, more preferably may not exceed 40 mm2.

Depending on the application, the method also allows to generate straps which, in the implanted stage, exhibit an outer shape with a gradual outer transition between the section in which the front section is fastened to the rear section (e.g., by overlapping and suturing them), and the adjacent sections in which the front section is not fastened to the rear section (e.g., where the front section does not contact the rear section). For example, at a position of the strap in which the front section has been fastened to the rear section, the area occupied by the strap in a cross section orthogonal to the direction of extension of the spine between the first vertebra and the second vertebra may be less than 300%, preferably less than 250%, more preferably less than 200% of the cross sectional area occupied by the strap at a position adjacent to the position in which the front section has been fastened to the rear section.

### BRIEF DESCRIPTION OF SEVERAL VIEWS OF THE DRAWINGS

The invention described herein will be more fully understood from the detailed description given herein below and the accompanying drawings, which should not be considered limiting to the invention described in the appended claims. The drawings show:

FIG. 1 shows a visualization of the allograft preparation for both interlaminar vertebropexy (left) and interspinous vertebropexy (right) and determination of the graft length for the studies described in example 1;

FIG. 2A shows one illustration in a series of the interspinous vertebropexy in which the allograft is looped through the holes in the spinous processes and then tightened, as described in example 1;

FIG. 2B shows another illustration in the series of the interspinous vertebropexy in which the allograft is looped through the holes in the spinous processes and then tightened, as described in example 1;

FIG. 2C shows another illustration in the series of the interspinous vertebropexy in which the allograft is looped through the holes in the spinous processes and then tightened, as described in example 1;

FIG. 2D shows another illustration in the series of the interspinous vertebropexy in which the allograft is looped through the holes in the spinous processes and then tightened, as described in example 1;

FIG. 2E shows another illustration in the series of the interspinous vertebropexy in which the allograft is looped through the holes in the spinous processes and then tightened, as described in example 1;

FIG. 2F shows another illustration in the series of the interspinous vertebropexy in which the allograft is looped through the holes in the spinous processes and then tightened, as described in example 1;

FIG. 3A shows one illustration in a series of the interlaminar vertebropexy in which two allografts are passed behind the laminae and brought together to form a loop and are then tightened, as described in example 1;

FIG. 3B shows another illustration in the series of the interlaminar vertebropexy in which two allografts are passed behind the laminae and brought together to form a loop and are then tightened, as described in example 1;

FIG. 3C shows another illustration in the series of the interlaminar vertebropexy in which two allografts are passed behind the laminae and brought together to form a loop and are then tightened, as described in example 1;

FIG. 3D shows another illustration in the series of the interlaminar vertebropexy in which two allografts are passed behind the laminae and brought together to form a loop and are then tightened, as described in example 1;

FIG. 4A shows an illustration of the setup for biomechanical testing used in example 1;

FIG. 4B shows an illustration of the interspinous vertebropexy used in example 1;

FIG. 4C shows an illustration of the interlaminar vertebropexy as described in example 1;

FIG. 5 shows an illustration of the workflow of the experiments performed in example 1, wherein FE=flexion-extension, LB=lateral bending, AR=axial rotation, AS=anteroposterior shear, LS=lateral shear, ROM=range of motion;

FIG. 6A shows an illustration of the setup for biomechanical testing used in example 2;

FIG. 6B shows an illustration of the lateral view of the interspinous synthetic vertebropexy, as described in example 2;

FIG. 6C shows an illustration of the lateral view of the spinolaminar synthetic vertebropexy, as described in example 2;

FIG. 7A shows an illustration of interspinous vertebropexy after unilateral facetectomy using synthetic material, as described in example 2;

FIG. 7B shows an illustration of interspinous vertebropexy after unilateral facetectomy using synthetic material, as described in example 2;

FIG. 7C shows an illustration of interspinous vertebropexy after unilateral facetectomy using synthetic material, as described in example 2;

FIG. 8A shows an illustration of spinolaminar vertebropexy after unilateral facetectomy using synthetic material, as described in example 2;

FIG. 8B shows an illustration of spinolaminar vertebropexy after unilateral facetectomy using synthetic material, as described in example 2;

FIG. 8C shows an illustration of spinolaminar vertebropexy after unilateral facetectomy using synthetic material, as described in example 2;

FIG. 8D shows an illustration of spinolaminar vertebropexy after unilateral facetectomy using synthetic material, as described in example 2;

FIG. 8E shows an illustration of spinolaminar vertebropexy after unilateral facetectomy using synthetic material, as described in example 2;

FIG. 8F shows an illustration of spinolaminar vertebropexy after unilateral facetectomy using synthetic material, as described in example 2;

FIG. 9A shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9B shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9C shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9D shows the effect of microsurgical decompression, interspinous and spinolaminar fixation, and instrumentation; the y-axis shows the measured vertebral body segment range of motion (ROM) for different loading cases beyond the native state, as described in example 2;

FIG. 9E shows the effect of microsurgical decompression, interspinous and spinolaminar fixation, and instrumentation; the y-axis shows the measured vertebral body segment range of motion (ROM) for different loading cases beyond the native state, as described in example 2;

FIG. 10A shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10B shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10C shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10D shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 10E shows a comparison of the effect of interspinous fixation using a fibercerclage and ligamentous interspinous fixation (vertebropexy); the y-axis shows the measured vertebral body segment range of motion (ROM) for different loading cases beyond the native state, as described in example 2;

FIG. 11 shows the sample load deformation plots for each of the three graft types discussed in example 3 tested to failure;

FIG. 12 shows the sample load relaxation plot showing the difference in load between the initial load and the final load for each of the three graft types tested, as discussed in example 3;

FIG. 13 shows the sample creep plot showing the difference in displacement between the initial displacement and the final displacement for each of the 3 graft types tested, as discussed in example 3; and

FIG. 14 illustrates the force to strain curve of an exemplary strap made of a synthetic material, as discussed in example 3.

### DESCRIPTION OF PREFERRED EMBODIMENTS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

To illustrate variants of the invention, two related studies were performed, which are outlined in detail below. In the first study (see example 1), fifteen spinal segments were biomechanically tested in a stepwise surgical decompression and ligamentous stabilization study. Stabilization was achieved with a gracilis or semitendinosus tendon allograft, which was attached to the spinous process (interspinous vertebropexy) or the laminae (interlaminar vertebropexy) in form of a loop. The specimens were tested (1) in the native state, after (2) microsurgical decompression, (3) interspinous vertebropexy, (4) midline decompression, and (5) interlaminar vertebropexy. In the intact state and after every surgical step, the segments were loaded in flexion-extension (FE), lateral shear (LS), lateral bending (LB), anterior shear (AS) and axial rotation (AR). In the second study (see example 2), twelve spinal segments (Th12/L1: 4, L2/3:4, L4/5:4) were tested in a stepwise surgical decompression and stabilization study. Stabilization was achieved with a FiberTape cerclage, which was pulled through the spinous process (interspinous technique) or one spinous process and around both laminae (spinolaminar technique). The specimens were tested (1) in the native state, after (2) unilateral laminotomy, (3) interspinous vertebropexy and (4) spinolaminar vertebropexy. The segments were loaded in flexion-extension (FE), lateral shear (LS), lateral bending (LB), anterior shear (AS) and axial rotation (AR).

In the following, the two examples are described in detail.

### 1. FIRST EXAMPLE

### 1.1 Materials and Methods of Example 1

### a) Dissection, Preparation and Storage

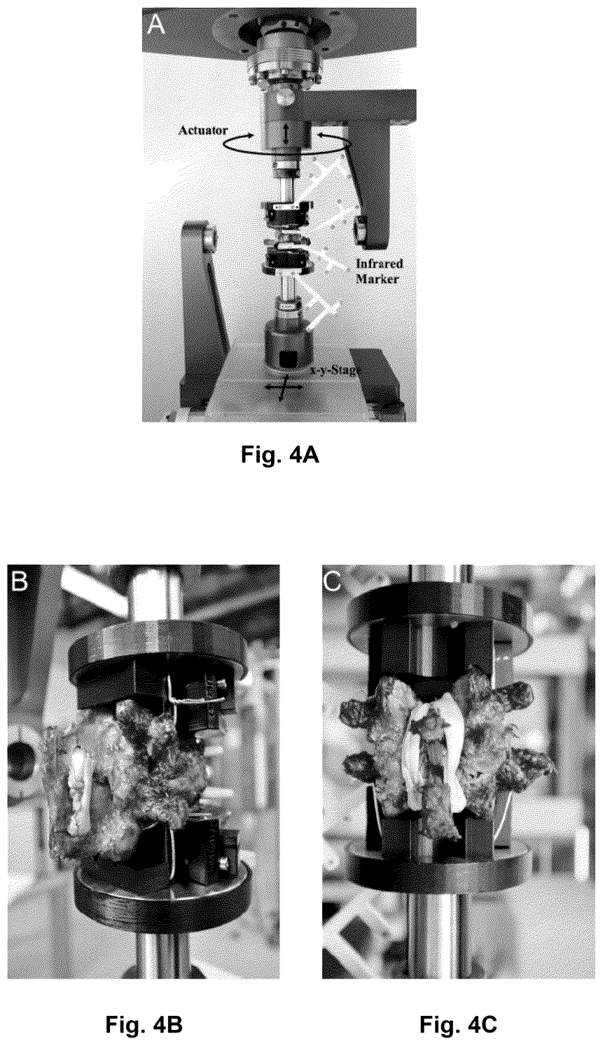

Fifteen spinal segments (TH12/L1: 3, L1/2: 3, L2/3: 3, L3/4: 3, L4/5: 3) originating from seven fresh frozen cadavers (Table 1; Science Care, Phoenix, AZ, USA) were tested. After thawing CT scans (SOMATOM Edge Plus, Siemens Healthcare GmbH, Erlangen, Germany) were performed to exclude bony defects. The specimens were carefully dissected without harming bony processes, paraspinal ligaments or the intervertebral discs. After preparation, the segments were mounted on a testing machine (FIG. 4A) with individualized 3D-printed-clamps [12].

| | | | | | Height | Weight | BMI |
|---|---|---|---|---|---|---|---|
| Specimen | Level | Sex | Age | Cause of death | (cm) | (kg) | (kg/m²) |
| C200862 | L1L2 | Male | 68 | Metastatic | 177.8 | 55.3 | 17.5 |
| | | | | malignancy of | | | |
| | | | | the stomach | | | |
| C200862 | L3L4 | Male | 68 | Metastatic | 177.8 | 55.3 | 17.5 |
| | | | | malignancy of | | | |
| | | | | the stomach | | | |
| S201932 | L1L2 | Female | 56 | Anoxic | 160 | 67.1 | 26.2 |
| | | | | brain failure | | | |
| S201932 | L3L4 | Female | 56 | Anoxic | 160 | 67.1 | 26.2 |
| | | | | brain failure | | | |
| S201942 | L1L2 | Male | 49 | Probable | 167.6 | 103.9 | 40 |
| | | | | atherosclerotic | | | |
| | | | | coronary disease | | | |
| S201942 | L3L4 | Male | 49 | Probable | 167.6 | 103.9 | 40 |
| | | | | atherosclerotic | | | |
| | | | | coronary disease | | | |
| S200838 | L4L5 | Male | 45 | Pending | 177.8 | 81.6 | 25.8 |
| S210555 | TH12L1 | Male | 57 | Acute | 182.9 | 68.5 | 20.5 |
| | | | | cardiac arrest | | | |
| S210555 | L2L3 | Male | 57 | Acute | 182.9 | 68.5 | 20.5 |
| | | | | cardiac arrest | | | |
| S210555 | L4L5 | Male | 57 | Acute | 182.9 | 68.5 | 20.5 |
| | | | | cardiac arrest | | | |
| L201826 | TH12L1 | Female | 62 | Acute | 167.6 | 132 | 47 |
| | | | | respiratory | | | |
| | | | | failure | | | |
| L201826 | L2L3 | Female | 62 | Acute | 167.6 | 132 | 47 |
| | | | | respiratory | | | |
| | | | | failure | | | |
| S210473 | TH12L1 | Male | 59 | Congestive | 167.6 | 66.2 | 23.6 |
| | | | | heart failure | | | |
| S210473 | L2L3 | Male | 59 | Congestive | 167.6 | 66.2 | 23.6 |
| | | | | heart failure | | | |
| S210473 | L4L5 | Male | 59 | Congestive | 167.6 | 66.2 | 23.6 |
| | | | | heart failure | | | |

### Table 1: Specimen Information

### b) Description of the Stepwise Surgical Decompression and Techniques of Vertebropexy

### Microsurgical Decompression and Interspinous Vertebropexy:

A bilateral approach was used with sparing laminotomy of the overlying and underlying lamina. Then a flavectomy was performed from cranial to caudal followed by a recessotomy in a standard fashion.

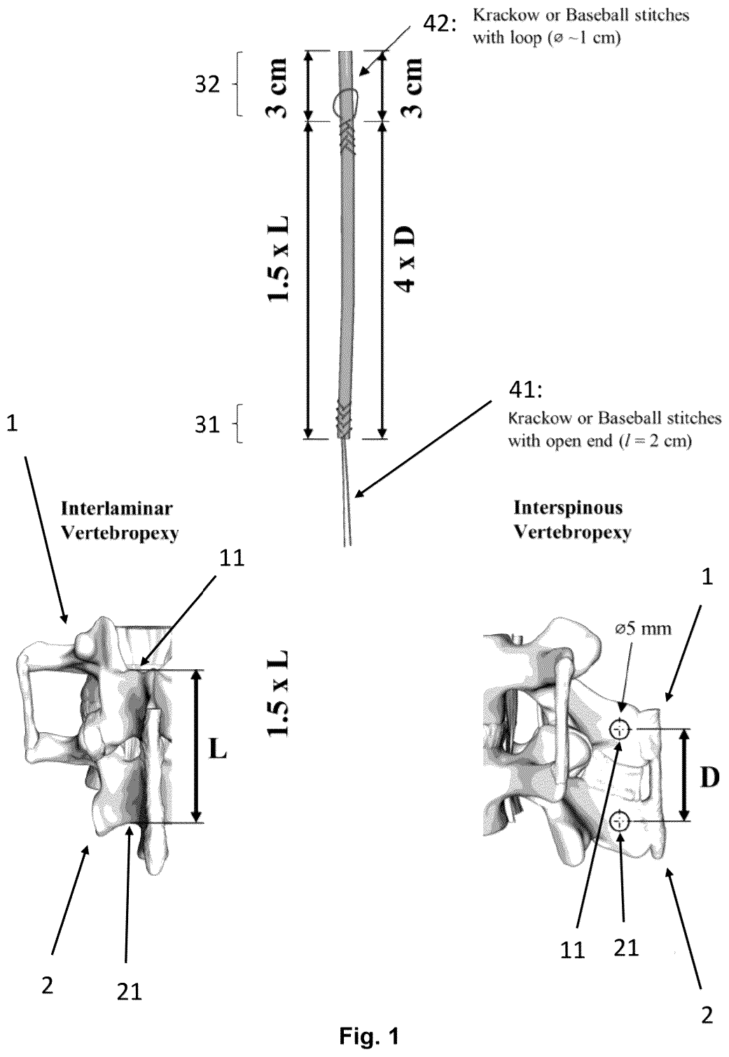

With reference to FIG. 1, both spinous processes were prepared for allograft passage by predrilling a 3.2-mm hole from one side to the other through the middle of the spinous process. The holes were overdrilled using a 5-mm drill bit, taking care not to create an iatrogenic fracture (cf. FIG. 1). A gracilis or semitendinosus tendon allograft (AlloSource, Centennial, Colorado) was prepared, thinning the allograft to a maximum diameter of 4 mm and reinforcing one end of the tendon with a Fiberwire No. 2 (Arthrex, Naples, Florida) using a 2-cm-long Krackow suture (FIG. 1; cf. Krackow K A, Cohn B T (1987) A new technique for passing tendon through bone. Brief note. J Bone Joint Surg Am Volume 69:922-4). For vertebropexy, the tendon graft was looped twice. The other end of the tendon was similarly reinforced with a Fiberwire No. 2, creating a loop in addition to the Krackow suture (FIG. 1).

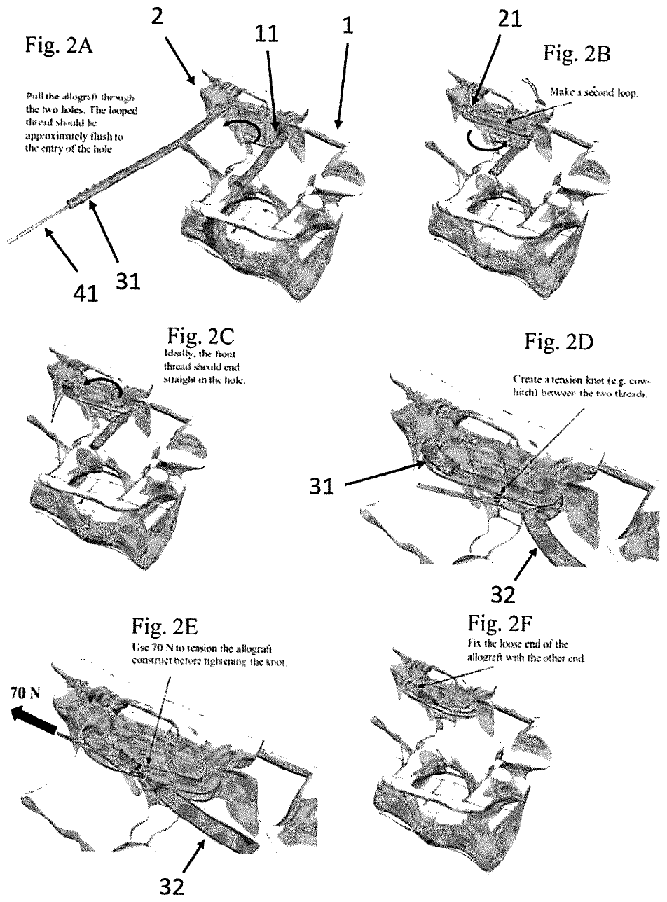

With reference to FIG. 2, thereafter, the allograft was pulled through the previously drilled holes in a double loop technique (FIG. 2A-C). An extension load of 5 Nm was applied via the static testing machine to simulate a prone position with physiological extension of the lumbar spine. The Fiberwire was knotted using the cow hitch technique (FIG. 2D): a double-stranded stranded knot configuration with a loop on one side, secured by four half hitches. This technique is biomechanically stronger and stiffer compared to several other conventional knots (cf. Meyer D C, Bachmann E, Lädermann A, et al (2018) The best knot and suture configurations for high-strength suture material. An in vitro biomechanical study. Orthop Traumatol Surg Res 104:1277-1282). The knot was tightened with a force of 70N using a needle holder (FIG. 2E). The applied force was objectified with a force gauge. Finally, the second end of the tendon was sutured to the loop (FIG. 2F, FIG. 4B).

The same surgical approach is used for both steps (decompression and fixation) so no additional muscle attachments need to be released for fixation of the vertebral segment.

### Midline Decompression and Interlaminar Vertebropexy:

The supraspinous and interspinous ligaments were sharply removed with a Leksell rongeur, also the two spinous processes were partially removed. Mid-line decompression with the osteotome was performed, while care was taken not to harm the facet joints. The remaining ligamentum flavum was exposed and removed from cranial to caudal.

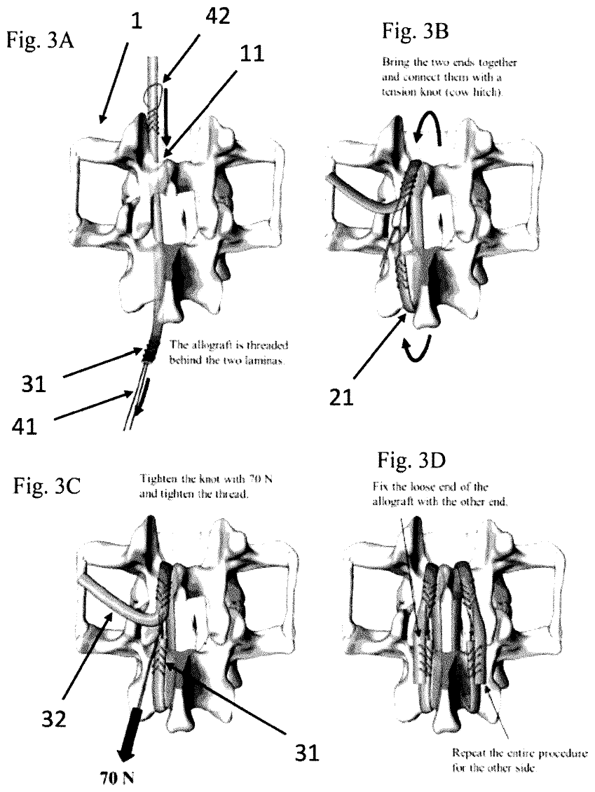

Two tendon allografts were reinforced in the same manner as described above (FIG. 1). With reference to FIG. 3, then the reinforced ends of the tendons were carefully passed under the laminae on both sides from cranial to caudal (FIG. 3A). A rongeur was used to pull the tips of both tendons up through the distal interlaminar window or above the inferior lamina (FIG. 3B). The segment was reloaded with 5 Nm extension and the Fiberwire was knotted bilaterally using a cow hitch and tightened with a force of 70 N (FIG. 3C). The remaining part of the tendon was sutured to the loop (FIG. 3D, FIG. 4C).

### c) Biomechanical Experiments

With reference to FIG. 4, biomechanical testing of the 15 specimens was performed on a biaxial (linear & torsion) static testing machine (Zwick/Roell Allroundline 10 kN and testXpert III Software, ZwickRoell GmbH & Co. KG, Germany; FIG. 4A). The system is based on a traverse to generate vertical compression and tension and a torsion motor to generate torque in the horizontal plane. The machine was complemented with a testing setup consisting of an x-y-table and holding arms that allow for specimen fixation in a horizontal orientation for flexion-extension (FE), lateral shear (LS), lateral bending (LB), and anteroposterior shear (AS), and in a vertical orientation for axial rotation (AR). A customized mounting apparatus for the clamped specimens was used (cf. Cornaz F, Fasser M-R, Spirig J M, et al (2019) 3D Printed Clamps Improve Spine Specimen Fixation in Biomechanical Testing. J Biomech 98:109467), consisting of high-precision fitting rings, pins, and a mechanism to compress the connection with a defined load before tightening. Loading was applied to the cranial vertebra while the caudal vertebra was fixed to the x-y-table allowing for translational movement orthogonal to the loading direction. Coupled motions around the x-and y-axis were prevented, restricting all motions to the test plane. With this configuration, translation forces, as might occur with a fully constrained setup, are eliminated, resulting in pure moments and pure forces in the plane of interest. Further details on the test setup, including images of all loading configurations, are provided in a previously published study, which is incorporated herein by reference: Widmer J, Cornaz F, Scheibler G, et al (2020) Biomechanical contribution of spinal structures to stability of the lumbar spine-novel biomechanical insights. Spine J 20:1705-1716.

### d) Biomechanical Testing Protocol

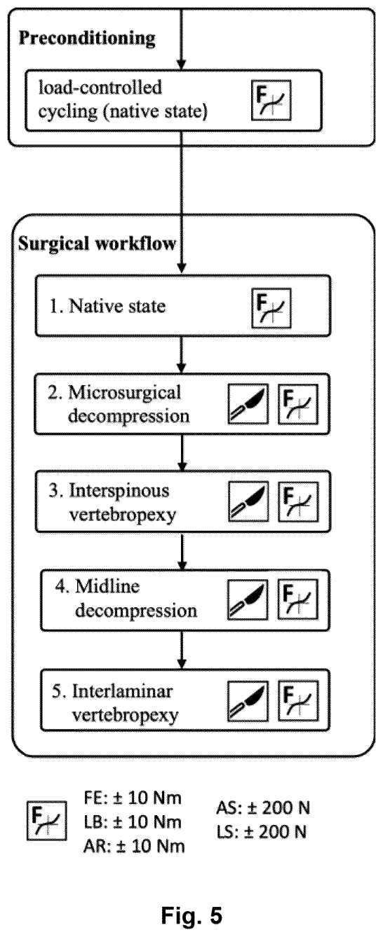

With reference to FIG. 5, each specimen was tested load-controlled (1) in the native state, after (2) microsurgical decompression, (3) interspinous vertebropexy, (4) midline decompression and (5) interlaminar vertebropexy. The surgical steps are illustrated in FIG. 5. After every surgical step, the segments were loaded in FE, LS, LB, AS and AR (in the listed order). For each loading case, 5 preloading cycles were conducted before the relative motion between the cranial and caudal vertebral bodies was recorded in the sixth cycle.

The segments were initially loaded with ±10 Nm in the bending planes and ±200 N in shear loading. In order to test the fixation techniques on extreme loads, slightly higher loads were chosen than the physiological range. Loading was applied with a velocity of 1°/sec in flexion-extension and lateral bending, 0.5°/sec in axial rotation, and 0.5 mm/sec in anterior, posterior and lateral shear (cf. Wilke H-J, Wenger K, Claes L (1998) Testing criteria for spinal implants: recommendations for the standardization of in vitro stability testing of spinal implants. Eur Spine J 7:148-154). During testing, specimens were kept moist by frequently spraying them with phosphate buffered saline.

### e) Data Analysis

The 3D motion data of the vertebrae (Atracsys Fusion Track 500, recording frequency 10 Hz, tracking accuracy 0.09 mm [RMS]) were used to correct the load-deflection curves of the testing machine. The centerline of the load-deflection hysteresis was fitted using a fifth-order polynomial. A standardized method (for further details, see the following publication, which is incorporated herein by reference: Widmer J, Cornaz F, Scheibler G, et al (2020) Biomechanical contribution of spinal structures to stability of the lumbar spine-novel biomechanical insights. Spine J 20:1705-1716) was used to separate positive/negative load directions in the load-deflection curves. For symmetrical load directions (LB, AR, and LS), the average values between negative and positive load (left, right) were used. Torsional preload in the sagittal plane was determined by analyzing the moment change in the neutral position between flexion and extension after each surgical step.

The statistical evaluation was performed with MATLAB (Matlab 2020b, Math-Works, Massachusetts, USA). The difference in range of motion (ROM) relative to the native condition is reported with median and interquartile range. The Wilcoxon signed rank test was used for the statistical comparison of matched relative ROM values. Specifically, for the obtained results in each of the five loading directions, the ROM after the vertebropexies was compared with the movement after the respective previous decompression steps and a third comparison consists of the assessment of possible differences between the two vertebropexy steps. The mean values were used for segment-wise analysis, as only three data points were available per spinal level. Due to multiple comparisons, the significance level a was adjusted with Bonferroni corrections and set to be 0.05/2=0.025.

### 1.2 Results

The absolute ROM of the native segment and the segment after microsurgical decompression, interspinous vertebropexy, midline decompression, and inter-laminar vertebropexy is shown in Table 2 according to loading case. The table shows the absolute mean range of motion native, after surgical decompression and stabilization by segment.

| | | Microsurgical | | Midline | Interlaminar |
|---|---|---|---|---|---|
| | Native | decompression | Vertebropexy | decompression | vertebropexy |
| Th12/L1 | FE 5.7 | FE 5.9 | FE 2.2 | FE 6.4 | FE 2.7 |
| | LS 0.6 | LS 0.7 | LS 0.5 | LS 0.7 | LS 0.6 |
| | LB 5 | LB 5.2 | LB 5.2 | LB 5.6 | LB 5.5 |
| | AS 0.7 | AS 0.8 | AS 0.8 | AS 0.9 | AS 0.9 |
| | AR 1.5 | AR 1.5 | AR 1.5 | AR 1.8 | AR 1.8 |
| L1/L2 | FE 5.8 | FE 6.3 | FE 4 | FE 6.7 | FE 2.7 |
| | LS 1.8 | LS 2 | LS 1.9 | LS 2.1 | LS 1.8 |
| | LB 5.4 | LB 5.6 | LB 5 | LB 6 | LB 5.7 |
| | AS 2.3 | AS 2.7 | AS 2.5 | AS 2.9 | AS 2.6 |
| | AR 1.6 | AR 1.7 | AR 1.6 | AR 2.4 | AR 2.4 |
| L2/L3 | FE 9.2 | FE 9.6 | FE 2.7 | FE 10.6 | FE 4.3 |
| | LS 1.4 | LS 1.5 | LS 1 | LS 1.6 | LS 1.2 |
| | LB 10.1 | LB 10.4 | LB 9.6 | LB 11 | LB 9.8 |
| | AS 2.2 | AS 2.3 | AS 1.9 | AS 2.6 | AS 2.2 |
| | AR 3.7 | AR 3.8 | AR 3.1 | AR 3.9 | AR 3.3 |
| L3/L4 | FE 9.2 | FE 9.9 | FE 3.6 | FE 11.8 | FE 4.1 |
| | LS 3.6 | LS 3.7 | LS 3.3 | LS 4.6 | LS 3.9 |
| | LB 8.8 | LB 9.2 | LB 8 | LB 10.7 | LB 9.4 |
| | AS 3.6 | AS 3.9 | AS 3.5 | AS 5.2 | AS 4.5 |
| | AR 7.4 | AR 7.6 | AR 7 | AR 9.2 | AR 8.6 |
| L4/L5 | FE 12.2 | FE 12.6 | FE 4 | FE 13.1 | FE 4.7 |
| | LS 1.9 | LS 1.9 | LS 1.4 | LS 2 | LS 1.5 |
| | LB 9.3 | LB 9.7 | LB 9 | LB 10.1 | LB 9.3 |
| | AS 1.7 | AS 1.9 | AS 1.6 | AS 2 | AS 1.8 |
| | AR 3.7 | AR 3.8 | AR 3.3 | AR 3.8 | AR 3.6 |

Table 2: for each of the five levels (TH12/L1, L1/2, L2/3, L3/4, L4/5) three cadaveric segments were available (15 segments in total); FE: flexion-extension (°); LS: lateral shear (mm); LB: lateral bending (°); AS: anteroposterior shear (mm); AR: axial rotation (°).

### a) Interspinous Vertebropexy

Interspinous vertebropexy significantly reduced the ROM in all loading scenarios. The ligamentous stabilization technique was able to decrease the ROM after microsurgical decompression in FE by almost 70% (p<0.001), in LS by 22% (p<0.001), in LB by 8% (p<0.001), in AS by 12% (p<0.01), and in AR by 9% (p<0.001). The effect of interspinous vertebropexy based on segments (see table 2 above) was relatively constant, and independent of the segment.

### b) Interlaminar Vertebropexy

Interlaminar vertebropexy significantly decreased the ROM of all segments compared to midline decompression in all loading scenarios. ROM was decreased by 70% (p<0.001) in FE, 18% (p<0.001) in LS, 11% (p<0.01) in LB, 7% (p<0.01) in AS, and 4% (p<0.01) in AR. The ROM was comparable between the segments.

### c) Comparison of the Two Vertebropexy Techniques

Vertebral segment ROM was significantly smaller with the interspinous vertebropexy compared to the interlaminar vertebropexy for all loading scenarios except FE. In FE, the effect of the two techniques was comparable (36.7% vs. 43.1%, p=0.08 (median; relative ROM after stabilization; native=100%). Significantly smaller vertebral segment ROM was achieved using interspinous vertebropexy in LS (81.7% vs. 98.1%; p<0.01), LB (95.9% vs. 100.3%; p<0.001), AS (96.3% vs. 115.9%; p<0.001), and AR (93.5% vs. 115.5%; p<0.001).

Overall, both techniques decreased vertebral body segment ROM in FE, LS and LB beyond the native state. The vertebropexy mainly reduced ROM in FE, in the other loading cases the effect was considerably smaller. The decompression steps led to increased ROM in each loading scenario compared to the native state.

### 2. SECOND EXAMPLE

### 2.1 Materials and Methods of Example 2

### a) Dissection, Preparation and Storage

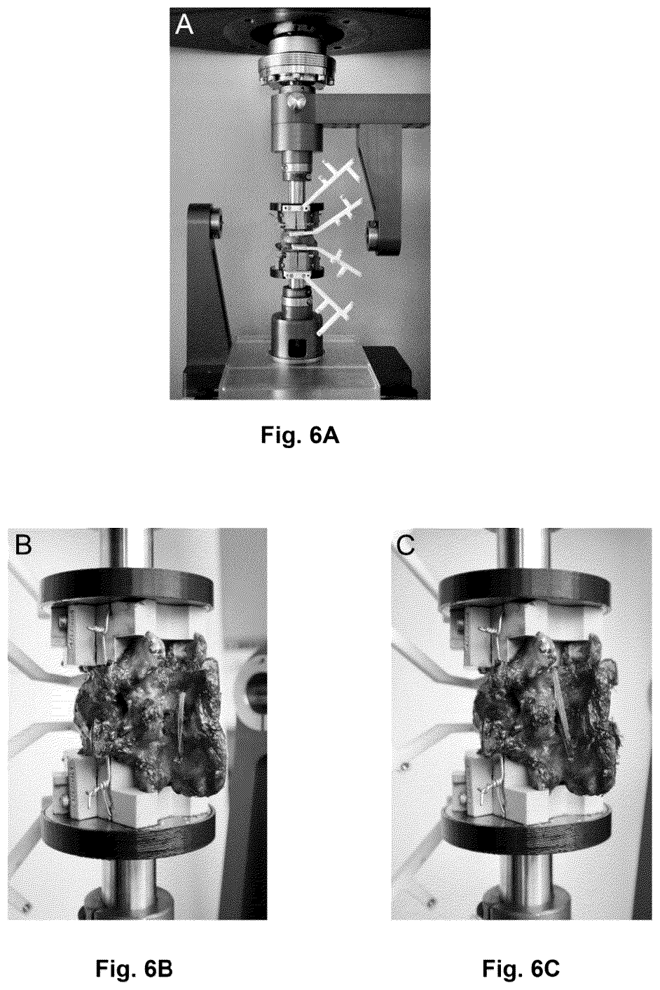

Twelve spinal segments (Th12/L1: 4, L2/3: 4, L4/5: 4) originating from five fresh frozen cadavers (Table 3 below; Science Care, Phoenix, AZ, USA) were tested. Except for age-appropriate changes, the specimens were free of any osseous defects or deformities based on computed tomography scans (SOMATOM Edge Plus, Siemens Healthcare GmbH, Erlangen, Germany). After thawing, the cadavers were each separated into the vertebral segments Th12-L1, L2-L3, and L4-L5. The specimens were denuded of the surrounding muscle and connective tissue without harming the intersegmental ligamentous structures, facet joints, or intervertebral discs. After preparation, the segments were mounted on a testing machine (FIG. 6) with individualized 3D-printed-clamps (cf. Cornaz F, Fasser M-R, Spirig J M, et al (2019) 3D Printed Clamps Improve Spine Specimen Fixation in Biomechanical Testing. J Biomech 98:109467).

TABLE 1
Specimen information

| | | | | | Height | Weight | BMI |
|---|---|---|---|---|---|---|---|
| Specimen | Level | Sex | Age | Cause of death | (cm) | (kg) | (kg/m²) |
| C220688 | TH12L1 | Male | 75 | Acute hypoxic | 180.3 | 68 | 20.9 |
| | | | | and hypercapnic | | | |
| | | | | respiratory failure | | | |
| C220688 | L2L3 | Male | 75 | Acute hypoxic | 180.3 | 68 | 20.9 |
| | | | | and hypercapnic | | | |
| | | | | respiratory failure | | | |
| C220707 | L4L5 | Female | 94 | COPD | 154.9 | 35.3 | 14.7 |
| L200232 | TH12L1 | Male | 71 | Cardiorespiratory | 167.6 | 150.1 | 53.4 |
| | | | | arrest | | | |
| L200232 | L2L3 | Male | 71 | Cardiorespiratory | 167.6 | 150.1 | 53.4 |
| | | | | arrest | | | |
| L200232 | L4L5 | Male | 71 | Cardiorespiratory | 167.6 | 150.1 | 53.4 |
| | | | | arrest | | | |
| L211459 | TH12L1 | Female | 78 | COPD, tobacco use | 170.2 | 93.4 | 32.3 |
| L211459 | L2L3 | Female | 78 | COPD, tobacco use | 170.2 | 93.4 | 32.3 |
| L211459 | L4L5 | Female | 78 | COPD, tobacco use | 170.2 | 93.4 | 32.3 |
| P220110 | TH12L1 | Female | 43 | Metastatic rectal | 160 | 68.9 | 26.9 |
| | | | | adenocarcinoma | | | |
| P220110 | L2L3 | Female | 43 | Metastatic rectal | 160 | 68.9 | 26.9 |
| | | | | adenocarcinoma | | | |
| P220110 | L4L5 | Female | 43 | Metastatic rectal | 160 | 68.9 | 26.9 |
| | | | | adenocarcinoma | | | |

### Table 3: Specimen Information

### b) Description of the Stepwise Surgical Decompression and Techniques of the Synthetic Vertebropexies

Microsurgical Decompression with Unilateral Laminotomy and Interspinous Synthetic Vertebropexy:

A unilateral approach was used with sparing laminotomy of the overlying and underlying lamina. Then a flavectomy was performed from cranial to caudal followed by a recessotomy in a standard fashion.

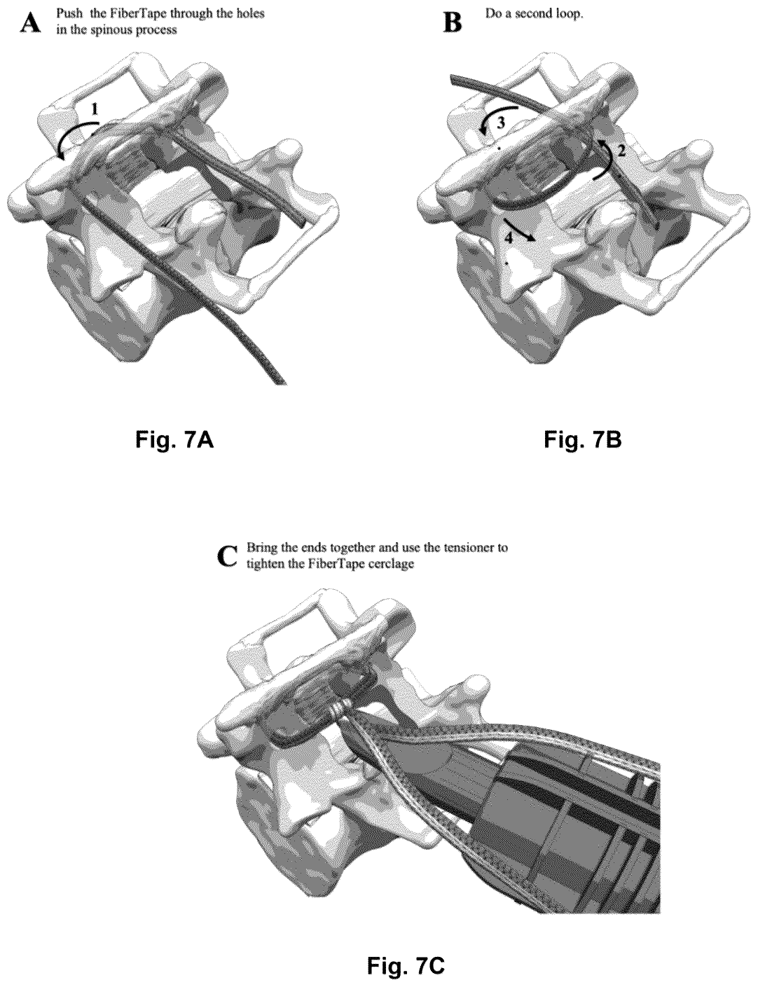

With reference to FIG. 7, for interspinous fixation, the technique of interspinous vertebropexy was followed, with the exception that synthetic material was used in the present biomechanical tests. Both spinous processes were prepared by drilling a 3.2-mm hole from one side to the other through the middle of the spinous process (FIG. 7). A FiberTape Cerclage (Arthrex, Naples, Florida) was pulled through the previously drilled holes in a double loop technique (FIG. 7). An extension load of 5 Nm was applied via the static testing machine to simulate a prone position with physiological extension of the lumbar spine. The cerclage was then tightened in a standardized manner with the corresponding tensioner, applying a force of approximately 40 pounds in each case (corresponds to the second marking on the tensioner). Afterwards, the cerclage was secured with five knots, using the tensioner to tighten the first knot.

### Spinolaminar Synthetic Vertebropexy:

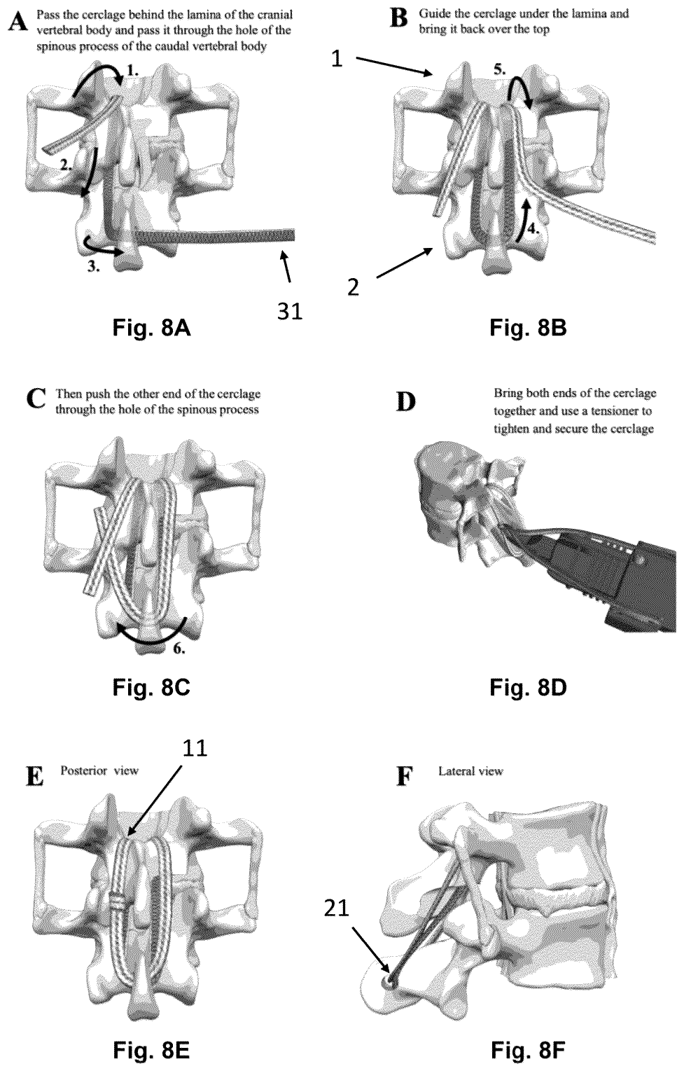

With reference to FIG. 8, a FiberTape cerclage was first passed through the pre-existing hole in the spinous process of the distal vertebra and then passed cranially anterior of the lamina of the proximal. The cerclage was then looped around the lamina and passed again through the hole in the spinous process of the distal vertebra. The same procedure was followed on the opposite side of the vertebra (FIG. 8). The FiberTape cerclage was then tightened as described above and secured with five knots.

### c) Biomechanical Experiments

Biomechanical testing of the twelve specimens was performed on a biaxial (linear and torsion) static testing machine (Zwick/Roell Allroundline 10 kN and testXpert III Software, ZwickRoell GmbH & Co. KG, Germany; FIG. 1). The system is based on a traverse: vertical compression and tension can be generated, and torque can be generated in the horizontal plane using a torsion motor. The machine was complemented with a test setup. It consisted of an x-y table and holding arms, allowing specimens to be fixed in horizontal orientation for flexion-extension (FE), lateral shear (LS), lateral bending (LB) and anteroposterior shear (AS), and in vertical orientation for axial rotation (AR). A customized mounting jig was used for the clamped specimens (cf. Cornaz F, Fasser M-R, Spirig J M, et al (2019) 3D Printed Clamps Improve Spine Specimen Fixation in Biomechanical Testing. J Biomech 98:109467). In each case, the cranial vertebra was loaded while the caudal vertebra was fixed on the x-y table. This allowed translational motion orthogonal to the loading direction, generating pure bending moments and shear forces. The setup allowed specimen fixation with extremely high reproducibility (variability <0.005°) (cf. Widmer J, Cornaz F, Scheibler G, et al (2020) Biomechanical contribution of spinal structures to stability of the lumbar spine-novel biomechanical insights. Spine J 20:1705-1716).

### d) Biomechanical Testing Protocol

Each specimen was tested load-controlled (1) in the native state, after (2) unilateral laminotomy, (3) interspinous vertebropexy and (4) spinolaminar vertebropexy. After each surgical step, the segments were loaded in FE, LS, LB, AS, and AR (in the order listed). For each loading case, five preloading cycles were performed before recording the relative motion between the cranial and caudal vertebral bodies in the sixth cycle. Data were recorded throughout the loading cycle, and the amplitude of translational motion of the markers (LS, AS) and projected angulation in the plane of motion (FE, LB, AR) were evaluated.

The segments were initially loaded with ±10 Nm in the bending planes and ±200 N in the shear loading. Slightly higher loads than in the physiological range were chosen to test the fixation techniques at extreme loading. Loading was applied at a rate of 1°/sec for flexion-extension and lateral bending, 0.5°/sec for axial rotation, and 0.5 mm/sec for anterior, posterior, and lateral shear (cf. Cornaz F, Widmer J, Farshad-Amacker N A, et al (2020) Biomechanical Contributions of Spinal Structures with DifferentDegrees of Disc Degeneration. Spine 46: E869-E877). During testing, specimens were kept moist by frequent spraying with phosphate-buffered saline.

The following comparisons of segmental ROM were undertaken: (1) microsurgical decompression with unilateral laminotomy versus synthetic vertebropexies, (2) synthetic interspinous versus spinolaminar vertebropexy, (3) ligamentous interspinous vertebropexy versus synthetic interspinous vertebropexy, and (4) synthetic vertebropexies versus dorsal fusion. For this purpose, data sets from the previously published studies were used, which are incorporated herein by reference: Farshad M, Burkhard M D, Spirig J M (2021) Occipitopexy as a Fusionless Solution for Dropped Head Syndrome: A Case Report. JBJS Case Connect 11 (3): e21.00049.

### e) Data Analysis

The statistical evaluation was performed with MATLAB (Matlab 2020b, Math-Works, Massachusetts, USA). The difference in range of motion (ROM) relative to the native condition is reported with the median and interquartile range. The Wilcoxon signed rank test was used for the statistical comparison of matched relative ROM values. Specifically, for the obtained results in each of the five loading directions, the relative ROM after the synthetic vertebropexies was compared with the movement after microsurgical decompression with unilateral laminotomy and a third comparison consisted of the assessment of possible differences between the two synthetic vertebropexies. Unpaired comparisons of the relative ROM after the synthetic vertebropexies and measurements of the same parameter after microsurgical decompression and instrumentation were performed with Wilcoxon rank sum tests. Furthermore, interspinous synthetic vertebropexy was compared with the ROM after interspinous ligamentous vertebropexy. The significance level alpha was set to 0.05 and the p-values were corrected according to Bonferroni to adjust for multiple comparisons.

### 1.2 Results

a) Effect of Synthetic Vertebropexies After Microsurgical Decompression with Unilateral Laminotomy

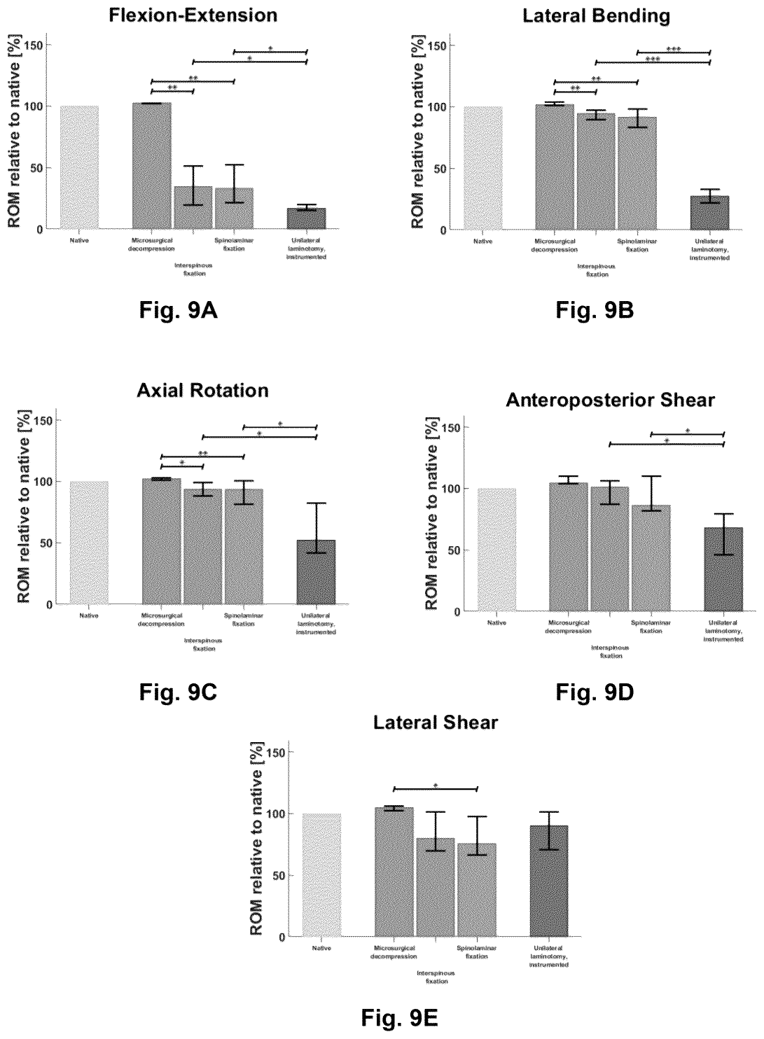

With reference to FIG. 9, microsurgical decompression increased native ROM in all loading cases (FIG. 9): in FE by 2%, in LS by 5%, in LB by 1%, in AS by 4%, and in AR by 2%.

Interspinous fixation significantly reduced ROM after microsurgical decompression in FE by 66% (p=0.003), in LB by 7% (p=0.006), and in AR by 9% (p=0.02). Shear movements (LS and AS) were also reduced, although not significantly: in LS reduction by 24% (p=0.07), in AS reduction by 3% (p=0.21).

Spinolaminar fixation significantly reduced ROM after microsurgical decompression in FE by 68% (p=0.003), in LS by 28% (p=0.01), in LB by 10% (p=0.003), and AR by 8% (p=0.003). AS was also reduced, although not significantly: reduction by 18% (p=0.06).

### b) Comparison of Interspinous and Spinolaminar Vertebropexy Using Synthetic Material

With reference to FIG. 9, the effect of the two techniques was comparable: FE 34.6% vs. 32.9%, p=1 (median; relative ROM after interspinous versus spinolaminar fixation; native=100%); LS 79.9% vs. 75.2%, p=1; LB 94% vs. 91.2%, p=1; AS 100.8% vs. 86%, p=1; and AR 93.1% vs. 93.3%, p=1.

Overall, both techniques decreased vertebral body segment ROM in all loading cases beyond the native state, except for the interspinous fixation technique, which only slightly influenced AS movement.

The spinolaminar technique had a higher effect on shear motion compared to interspinous fixation. Overall, both techniques mainly influenced ROM in FE.

### c) Comparison of Ligamentous Interspinous Fixation (Ligamentous Vertebropexy) and Interspinous Fixation Using a Fibercerclage (Synthetic Vertebropexy)

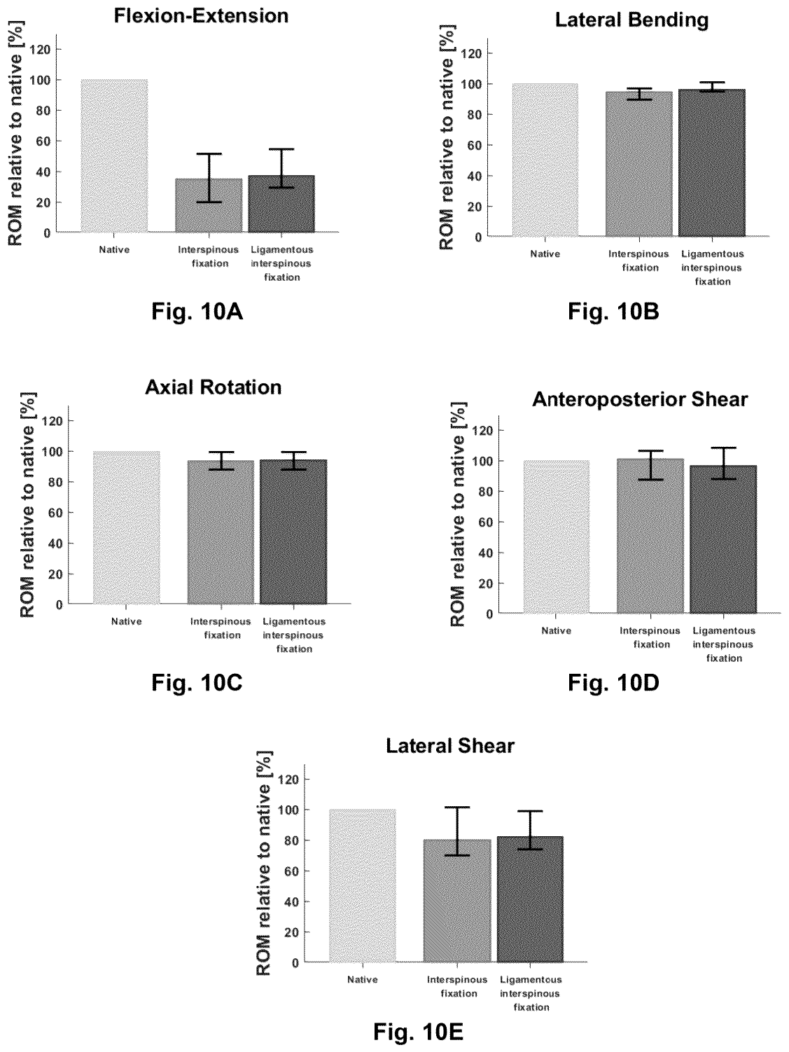

With reference to FIG. 10, the effect of the two techniques was comparable and thus largely independent of the material used for stabilization: FE 34.6% vs. 36.8%, p=1 (median; relative ROM after interspinous versus ligamentous interspinous fixation; native=100%); LS 79.9% vs. 81.7%, p=1; LB 94% vs. 95.9%, p=0.9; AS 100.8% vs. 96.3%, p=1; and AR 93.1% vs. 93.5%, p=1.

### d) Comparison of Synthetic Vertebropexies and Dorsal Fusion

With reference to FIG. 9, both synthetic vertebropexies affected all loading cases, but significantly less than fusion by connecting the inserted pedicle screws (FIG. 9). After fusion, all loading cases, except LS (LS 14% vs. 24% (p=1) vs. 28% (p=1)), showed significantly higher median relative reductions compared to interspinous and spinolaminar synthetic vertebropexy: FE 83.3% vs. 66% (median; relative reduction after fusion versus interspinous fixation, p=0.026) vs. 68% (relative reduction after fusion versus spinolaminar fixation, p=0.04); LB 73.3% vs. 7% (p<0.001) vs. 10% (p<0.001); AS 34.9% vs. 3% (p=0.02) vs. 18% (p=0.02); and AR 49% vs. 9% (p=0.02) vs. 8% (p=0.02).

### 3. THIRD EXAMPLE

Example 3 describes the mechanical properties of some variants of the strap, as well as possible measurements to measure these mechanical properties. Only exemplary variants of the straps are discussed in example 3. Other variants of the strap may also be envisioned.

### 3.1 Part 1: Grafts

In some variants, the strap may comprise or consist of a graft. Different grafts may be used. For example, a double-looped semitendinosus and gracilis (DLSTG) graft may be used. Depending on the application, a combination of semitendinosus and gracilis may be used in the form of a double loop, e.g., for interspinous vertebropexy, or the combination may be used in the form of two single loops, e.g., for interlaminar vertebropexy.

Part 1 or example 3 describes selected mechanical properties of exemplary grafts that may be used for the strap. Specifically, part 1 discusses anterior tibialis tendons, posterior tibialis tendons and a double-looped semitendinosus and gracilis (DLSTG) graft.

### Ramp-to-Failure Test

Double-loop grafts were clamped 95 mm from the steel bar with the liquid nitrogen freeze clamp at room temperature. The grafts were preconditioned between 20 N and 250 N by applying 10 cycles at 0.1 Hz; thereafter, a load of 20 N was maintained to set the initial gauge length until testing (note: this means that the origin of the curve at which displacement/strain=0 was placed at 20 N load). The load-to-failure test was performed 15 minutes after preconditioning by pulling the graft to failure at a strain rate of 2%/s (1.5 mm/s). Table 4 below shows a comparison of the structural properties between the anterior tibialis and DLSTG graft and the posterior tibialis and DLSTG graft for 95-mm graft length (mean±SD).

TABLE 4

| | Ultimate | Linear | Ultimate | |
|---|---|---|---|---|
| | Load | Stiffness | Displacement | Area |
| Type of Graft | (N) | (N/mm) | (mm) | (mm²) |
| Anterior | 4,122 ± 893* | 460 ± 101 | 12.0 ± 3.0* | 48.2 ± 11.8 |
| Tibialis | (P = .005) | (NS, P = .283) | (P = .007) | (NS, P = .432) |
| Posterior | 3,594 ± 1,330 | 379 ± 143 | 12.5 ± 2.3* | 41.9 ± 17.3 |
| Tibialis | (NS. P = .204) | (NS, P = .467) | (P < .001) | (NS, P = .695) |
| DLSTG | 2,913 ± 645 | 418 ± 36 | 8.4 ± 1.3 | 44.4 ± 6.7 |

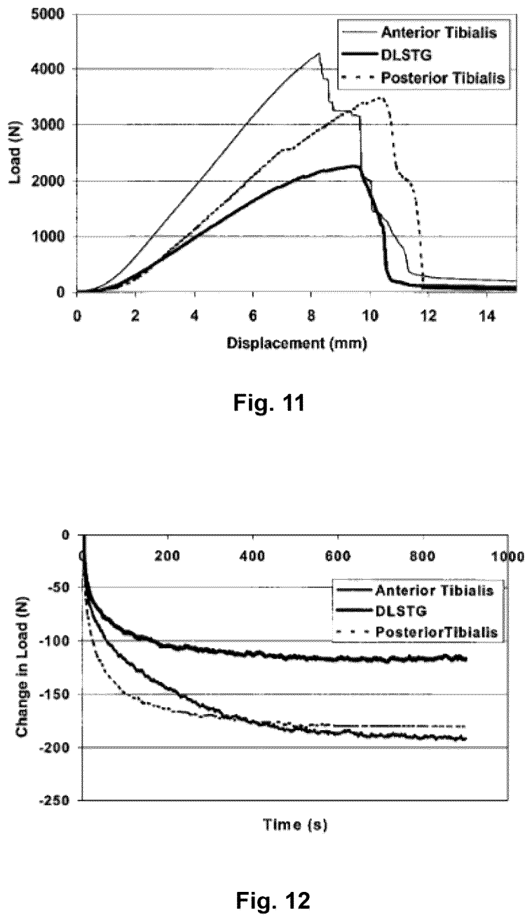

FIG. 11 shows the sample load deformation plots for each of the three graft types tested to failure. Stiffness and tensile modulus were determined in the linear region from 50% to 75% of the ultimate load. The ultimate load and stiffness of both single-loop tibialis tendon grafts were either similar to or greater than that of the DLSTG graft.

### Stress Relaxation Test

In a further test, double-loop grafts were clamped 75 mm from the steel bar with the liquid nitrogen freeze clamp. The test was performed 15 minutes after pre-conditioning. The test measured the decrease in load under a constant displacement (i.e., stress relaxation test) and was conducted by elongating the graft to, 2.5% strain at a rate of 250 mm/s. The load was recorded at 4 Hz while the displacement was held constant for either 15 minutes or until the load remained unchanged over 1 minute (i.e., less than 0.1% decrease in load).

FIG. 12 shows the sample load relaxation plot showing the difference in load between the initial load and the final load for each of the three graft types tested. Only the single-loop anterior tibialis tendon graft relaxed more than the DLSTG graft.

### Creep Test

In a further test, the graft was refrigerated overnight, and 24 hours later it was equilibrated to room temperature and preconditioned. The test was performed 15 minutes after preconditioning and measured the increase in displacement under a constant load (i.e., creep test) and was conducted by applying a 20 N load and increasing the load to 250 N at a rate of 315 N/s. The displacement was recorded at 1 Hz while the load was held constant at 250 N for either 15 minutes or until the displacement remained unchanged over 1 minute (i.e., less than a 0.1% increase in displacement.

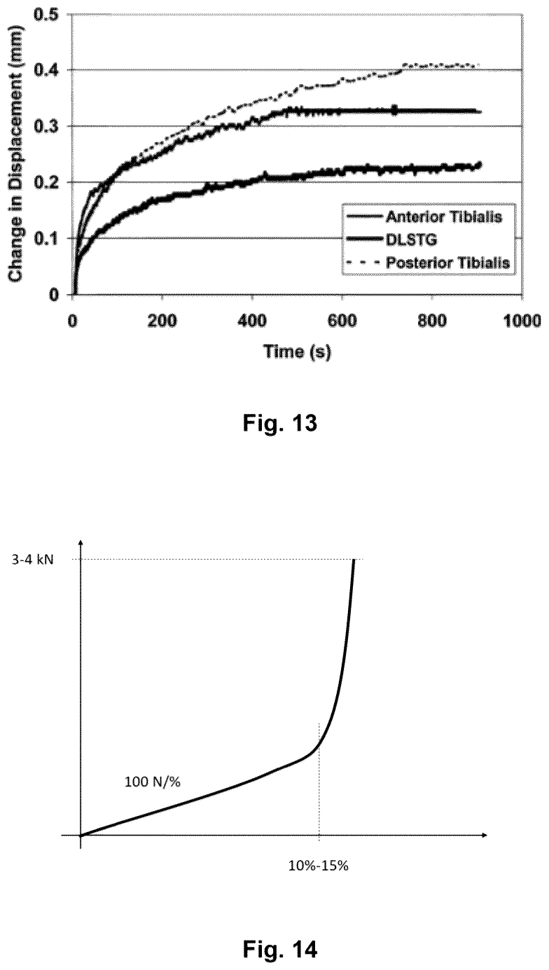

FIG. 13 shows the sample creep plot showing the difference in displacement between the initial displacement and the final displacement for each of the 3 graft types tested. Both single-loop tibialis tendon grafts crept more than the DLSTG graft, but the difference is not considered clinically important.

Table 5 shows a comparison of material properties between the anterior tibialis and DLSTG graft and the posterior tibialis and DLSTG graft (mean±SD).

TABLE 5

| | | Ultimate | Ultimate |
|---|---|---|---|
| Type of | Modulus | Stress | Strain |
| Graft | (MPa) | (MPa) | (%) |
| Anterior | 847 ± 301 | 89.8 ± 19.4* | 12.7 ± 3.2* |
| Tibialis | (NS, P = .618) | (P = .007) | (P = .006) |
| Posterior | 905 ± 230 | 89.1 ± 15.4* | 13.2 ± 2.4* |
| Tibialis | (NS, P = .983) | (P = .003) | (P < .001) |
| DLSTG | 904 ± 99 | 65.6 ± 12.0 | 8.8 ± 1.4 |

Abbreviation: NS, not significant.
*Denotes property significantly different from that of DLSTG graft.

Table 6 shows a comparison of viscoelastic properties between the anterior tibialis and DLSTG graft and the posterior tibialis and DLSTG graft for 75 mm graft length. Thus, depending on the application, the strap may have a decrease in load under a constant displacement from 100 N to 250 N, when measured with a strap length of 75 mm. Additionally, or alternatively, the strap may have an increase in displacement under a constant load from 0.1 mm to 0.6 mm, when measured with a strap length of 75 mm.

TABLE 6

| | Decrease in Load | Increase in |
|---|---|---|
| Type of | Under a Constant | Displacement Under |
| Graft | Displacement (N) | a Constant Load (mm) |
| Anterior | 215 ± 92* | 0.3 ± 0.1* |
| Tibialis | (P = .027) | (P = .004) |
| Posterior | 197 ± 91 | 0.4 ± 0.1* |
| Tibialis | (NS, P = .066) | (P < .001) |
| DLSTG | 134 ± 38 | 0.2 ± 0.0 |

Abbreviation: NS, not significant.
*Denotes property significantly different from that of DLSTG graft.

### 3.2 Part 2: Synthetic Material

In some variants, the strap may comprise or consist of a synthetic material. Depending on the application, the strap may have an elliptical cross-section. The cross-section may, for example, have a length from 8 mm to 12 mm and a height from 1 mm to 5 mm in a relaxed state.

FIG. 14 illustrates, for a diameter from 2 mm to 3 mm, the force to strain curve of an exemplary strap made of a synthetic material. A first section of the curve can be described as a linear response (Force to strain) with an inclination from 40 to 100 N per % strain. As an example, the inclination in the first section may be from 300 to 550 N/mm strain, preferably from 400 to 450 N/mm strain. In a second section of the curve, starting at a strain between 8 and 15% (yield point), the curve is steeper with an inclination from 500 to 1000 N per % strain. The ultimate tensile strength (failure force) may, for example, be from 3000 N to 4000 N. The ultimate tensile strain (displacement/strain of breaking point) may, for example, be from 17% to 20%. The % refers to the initial length of the ligament, possibly at preload 70N (force at which strain/displacement is set to 0).

It is claimed:

1. A method of stabilizing the spine of a patient in need thereof, comprising:
a. providing a first vertebra (1) having a first fastening edge (11) and a second vertebra (2) having a second fastening edge (21), wherein the first fastening edge (11) and the second fastening edge (21) are arranged opposite each other and facing away from each other;
b. providing a strap (3) extending in a longitudinal direction from a front section (31) to a rear section (32);
c. passing the front section (31) of the strap (3) around the first fastening edge (11), then from the first fastening edge (11) to the second fastening edge (21) and then around the second fastening edge (21);
d. optionally passing the front section (31) of the strap (3) from the second fastening edge (21) to the first fastening edge (11) and repeating step c;
e. tensioning the strap (3) such that the first vertebra (1) and the second vertebra (2) are fixated relative to each other;
f. fastening the front section (31) to the rear section (32) while the strap (3) is being tensioned.

2. The method according to claim 1, wherein the strap is hyperelastic.

3. The method according to claim 1, wherein the step of fastening the front section (31) to the rear section (31) includes at least one of knotting, suturing, sewing, stitching, knitting or felting the front section (31) to the rear section (31).

4. The method according to claim 1, wherein the strap (3) comprises a non-metallic cerclage, a ligament or a tendon, preferably a ligament.

5. The method according to claim 1, wherein the strap (3) comprises a front fastening fiber (41) connected to a main body of the strap (3) in or near the front section (31) and a rear fastening fiber (42) connected to the main body of the strap (3) in or near the rear section (31), wherein the rear fastening fiber (41) forms a loop configured for receiving the front fastening fiber (41).

6. The method according to claim 5, wherein the front fastening fiber (41) and/or the rear fastening fiber (41) are each connected to the main body of the strap (3) by a stitch, preferably a Krackow or Baseball stitch.

7. The method according to claim 5, wherein the step of tensioning the strap (3) comprises creating a tension knot between the front fastening fiber (41) and the rear fastening fiber (41), tensioning at least the front fastening fiber (41) and tightening the tension knot.

8. The method according to claim 1, wherein one of the two vertebrae is a cranial vertebra and the other of the two vertebrae is a caudal vertebra, wherein the fastening edge of the cranial vertebra is a cranial edge of the lamina and the fastening edge of the caudal vertebra is a caudal edge of the lamina.

9. The method according to claim 8, wherein the distance in the longitudinal direction between a front end of the front section (31) and a front end of the rear section (31) of the strap (3) in a relaxed state ranges from 120% to 180%, preferably from 140% to 160%, more preferably 150%, of the distance between the first fastening edge (11) and the second fastening edge (11).

10. The method according to claim 1, wherein one of the two vertebra is a cranial vertebra and the other of the two vertebra is a caudal vertebra, wherein the fastening edge of the cranial vertebra is a hole drilled in the spinous process of the cranial vertebra and the fastening edge of the caudal vertebra is a hole drilled in the spinous process of the caudal vertebra.

11. The method according to claim 10, wherein the distance in the longitudinal direction between a front end of the front section (31) and a front end of the rear section (31) of the strap (3) in a relaxed state ranges from 300% to 500%, preferably from 350% to 450%, more preferably from 380% to 420%, of the distance between the first fastening edge (11) and the second fastening edge (11).

12. The method according to claim 1, wherein after fastening the front section (31) to the rear section (31), the strap (3) exerts a tensile force on the first vertebra (1) and on the second vertebra (2) in a tensile force direction that is essentially in parallel to the direction of extension of the spine between the first vertebra (1) and the second vertebra (2).

13. The method according to claim 1, wherein one of the two vertebra is a cranial vertebra and the other of the two vertebra is a caudal vertebra, wherein the fastening edge of the cranial vertebra is a cranial edge of the lamina and the fastening edge of the caudal vertebra is a hole drilled in the spinous process of the caudal vertebra.

14. The method according to claim 1, wherein after fastening the front section (31) to the rear section (31), the strap (3) exerts a tensile force on the first vertebra (1) and on the second vertebra (2) in a tensile force direction that includes a vector component in the dorsal direction.

15. The method according to claim 1, wherein during the step of tensioning the strap (3), a tension force from 40 N to 90 N, preferably from 50 N to 70 N, is applied.

16. The method according to claim 1, wherein the step of fastening the front section (31) to the rear section (31) includes overlapping the front section (31) with the rear section (31) such that the front section (31) and the overlapped rear section (31) extend in opposite directions, and connecting the front section (31) with the overlapped rear section (31).

17. The method according to claim 1, wherein at a position of the strap (3) in which the front section (31) has been fastened to the rear section (31), the thread has a maximum width of up to 10 mm, preferably up to 8 mm, more preferably up to 6 mm, in at least one of the following directions: dorsal direction or lateral direction.

18. The method according to claim 1, wherein at a position of the strap (3) in which the front section (31) has been fastened to the rear section (31), the area occupied by the strap (3) in a cross section orthogonal to the direction of extension of the spine between the first vertebra (1) and the second vertebra (2) is less than 300%, preferably less than 250%, more preferably less than 200% of the cross sectional area occupied by the strap (3) at a position adjacent to the position in which the front section (31) has been fastened to the rear section (31).
